(19) United States
(12) Patent Application Publication
(10) Pub. No.: US 2012/0282297 A1
(43) Pub. Date: Nov. 8, 2012

(54) METHOD FOR TREATING CONSTIPATION

(76) Inventor: Eli D. Ehrenpreis, Skokie, IL (US)

(21) Appl. No.: 13/284,936

(22) Filed: Oct. 30, 2011

Related U.S. Application Data
(63) Continuation-in-part of application No. 12/399,778, filed on Mar. 6, 2009, now abandoned.
(60) Provisional application No. 61/041,233, filed on Mar. 31, 2008.

(51) Int. Cl. A61K 9/02; A61K 31/409; A61P 1/10

(52) U.S. Cl. 424/400; 514/410; 514/185

(57) ABSTRACT

The invention relates to methods and compositions for the treatment of constipation. The method of the invention involves administration of a suppository containing chlorophyll, chlorophyllin copper complex or chlorophyllin. The suppository contains an outer layer to prevent spillage of chlorophyll or chlorophyllin prior or after administration. Other ingredients that may also be effective treatments may be included. This method may be useful as a new and safer treatment for constipation, including constipation-predominant IBS and other functional anorectal disorders. Chlorophyll, or chlorophyll copper complex and chlorophyllin in the suppository form may improve bowel activity by stimulation of secretion and motility, thus relieving symptoms of constipation. This is the first description of this unique method of delivering these safe, natural products to patients suffering from constipation.

### CLAIM OF PRIORITY

This Application claims priority from U.S. Utility patent application Ser. No. 12/399,778 filed Mar. 6, 2009 entitled “METHOD FOR TREATING IRRITABLE BOWEL SYNDROME AND OTHER FUNCTIONAL GASTROINTESTINAL DISORDERS” which in turn claims priority from U.S. Provisional Patent Application Ser. No. 61/041,233 filed Mar. 31, 2008 entitled “METHOD FOR TREATING IRRITABLE BOWEL SYNDROME AND OTHER FUNCTIONAL GASTROINTESTINAL DISORDERS.”

### FIELD OF THE INVENTION

Disclosed is a method to alleviate the symptom of constipation using a new suppository

### BACKGROUND OF THE INVENTION

Constipation is a common symptom that occurs throughout the world. The estimated prevalence of constipation in the US population is between 12-19%. Constipation becomes more common with aging and is seen as a more frequent complaint in females compared to males. Irritable Bowel Syndrome (IBS), the most common functional gastrointestinal disorder, has been found to affect up to 20% of the population of the United States. Constipation is a cardinal symptom in about one third of patients with IBS. Additionally, certain animals, such as cats, dogs, and horses may be affected.

Constipation results in a large economic burden in the United States and other industrialized countries. For example, 2,500,000 visits to physicians for constipation occur annually in the US. Constipation also results in the expenditure of 6.9 billion dollars per year in the US and about $400,000,000 is spent annually in the US on laxatives.

Constipation may be a symptom of systemic diseases included endocrine disorders and neurologic disease. A large number of medications can cause constipation as a side effect. These include opioid analgesics, antihypertensives, antidepressants, antipsychotics, antiemetics and treatments for Parkinson's disease. Colonic and anorectal diseases, including carcinoma of the colon and rectum are associated with chronic constipation.

Once organic causes have been ruled out, patients with chronic constipation that do not have IBS are divided into three groups. Patients are defined as having functional constipation, constipation due to outlet obstruction and constipation due to slow colonic transit (also known as colonic inertia).

Constipation-Predominant IBS the term applied to patients with IBS and constipation. Many patients with IBS have abdominal discomfort and bloating as well as constipation.

Despite the variety of accessible treatments, refractory symptoms of constipation are not rare. Side effects are also common with many laxatives, limiting their effectiveness in some patients. The development of safe, naturally based treatments for constipation with limited toxicity is a high priority in the field of gastroenterology. Suppositories are bodies of solid materials into which medications have been incorporated. These medications are then placed into body cavities. Medications are released at the site of placement, resulting in local effects of the medications.

Suppository forms of medications are available for placement in the anus and vagina for the treatment of anorectal and gynecologic disorders. The most common use of rectal suppositories is for the treatment of constipation. Rectal suppositories are also used as an alternative form of drug delivery in patients that cannot receive medications by mouth. Examples of these types of rectal suppositories include treatments for nausea and pain.

Chlorophyll and chlorophyllin, the pigments utilized by plants to facilitate the conversion of carbon dioxide (CO2) to oxygen (O2) and create energy, has been used as a remedy for a variety of conditions. It has been used in an oral form as a folk remedy for halitosis, pancreatitis and reduction of colostomy odor. These effects have not been proven through scientific studies. No studies of the mechanisms of action of chlorophyll in humans have been performed. Chlorophyllin, the absorbable portion of chlorophyll has been used to reduce body odors. Only limited studies have been performed on gastrointestinal effects of chlorophyllin. Chlorophyllin, in the form of a non-absorbable copper complex, is available over-the counter from heath food stores in both liquid and capsule forms. Chlorophyll is available in capsules containing plant products such as alfalfa and capsules containing algae such as chlorella.

Although chlorophyll and chlorophyllin are folk remedies for some gastrointestinal symptoms including gaseous odor and bowel difficulties, little formal research has been performed to document their effectiveness. Clinical experience by the inventor suggests that some patients with constipation, alone or as a component of irritable bowel syndrome, may obtain mild symptomatic relief from oral chlorophyll therapy. However, oral chlorophyll and chlorophyllin appear to have mild laxative action, and relatively large oral doses of these appear to be necessary to produce this laxative effect. In addition, because oral chlorophyll and chlorophyllin probably work by softening the stool and perhaps by osmotic laxative effects, they would not be expected to be of much effect when given by oral route to patients with constipation that is due to poor functioning of the pelvic floor and anorectal muscles, as accumulation of the amount of chlorophyll in the rectum and sigmoid colon, required for evacuation in these patients would require large dosages of cholophyll. Direct application of chlorophyll in the form of a suppository circumvents this problem by allowing large doses of chlorophyll to concentrate in the rectum, resulting in direct stool softening and expulsion from the rectum.

### SUMMARY OF THE INVENTION

Disclosed is a method of treating constipation, including the steps of providing a suppository containing a pharmacologic dose of chlorophyll or chlorophyllin. The method further includes a step of placing the suppository within the rectal cavity for a period of time required for dissolution of the suppository. The suppository includes a soluble base and chlorophyll or chlorophyllin. The suppository containing chlorophyll or chlorophyllin is placed to reside within the rectal cavity for the time period required dissolution. Since the suppositories are fully dissolvable, release of chlorophyll or chlorophyllin from the suppository will be achieved after residence of the suppository in the rectum. The released chlorophyll or chlorophyllin will occur in high concentrations at the site of delivery, thus enhancing the effectiveness of this therapy for anorectal disorders.

The inventors have produced a suppository using chlorophyll using the following method to manufacture 40 suppositories containing 50 mg chlorophyll copper complex:

1) Suppository mold-EA suppository weighs approximately 2.18 mg

2) Weigh the following powders
a. Chlorophyll sodium-copper complex powder 2.0 gm
b. Polyethylene glycol 400 liquid 51.0 mL
c. Polyethylene glycol 8000 POS 34.0 gm

3) Wear gloves and cover all areas with disposable plastic or paper towels**this does not wash off most areas with how water**

4) Melt in microwave for 40 seconds-15 seconds-15 seconds (temperature should be under 130 degrees Fahrenheit)

5) Pour into molds and refrigerate

6) Finish by removing excess at top of mold

The outside coating (or shell) of the suppository is produced as follows:

1) Place 60 gm of cocoa butter in a heating pan

2) Heat to liquid at 122 degrees Fahrenheit

3) Cool cocoa butter to 94 degrees Fahrenheit

4) Place previously constructed chlorophyll copper complex suppositories in cooled cocoa butter for approximately 0.5 seconds.

5) Remove suppository immediately and place in suppository box and refrigerate.

According to a preferred embodiment, chlorophyll copper complex is contained within each suppository at a dose between 1 and 300 mg, combined within a polyethylene glycol suppository.

According to a further aspect of the invention, the anal suppository may further include thin coating (or shell) of cocoa butter, glycerin, wax or other fat-derived substance to prevent staining and leakage from chlorophyll contained within the suppository.

According to one embodiment, the constipation is due to outlet obstruction.

According to another embodiment, the constipation is due to chronic functional constipation.

According to another embodiment, the constipation is due to slow colonic transit (or colonic inertia)

According to another embodiment, the constipation is due to irritable bowel syndrome.

According to another embodiment, the constipation is due to an organic disease such as a neurologic or hormonal disease.

According to another embodiment, the constipation is due to medications.

In each of the aforementioned embodiments, the suppository may be constructed of chlorophyll, chlorophylline copper complex or chlorophyllin combined with polyethylene glycol.

In each of the aforementioned embodiments, the suppository may be constructed of chlorophyll, chlorophyllin copper complex or chlorophyllin combined with cocoa butter.

In each of the aforementioned embodiments, the suppository may be constructed of chlorophyll, cholorophyllin copper complex or chlorophyllin combined with olive oil.

In each of the aforementioned embodiments, the suppository may be constructed of chlorophyll, chlorophyllin copper complex or chlorophyllin combined with glycerin.

In each of the aforementioned embodiments, the suppository may be constructed of chlorophyll, chlorophyllin copper complex or chlorophyllin combined with any fat-derived substance.

In each of the aforementioned embodiments, the suppository may be constructed of chlorophyll, chlorophyllin copper complex or chlorophyllin combined with any ingredients required to produce a suppository.

In each of the aforementioned embodiments, the suppository may be constructed of chlorophyll, chlorophyllin copper complex or chlorophyllin combined with glycerin.

In each of the aforementioned embodiments, the suppository may be constructed of chlorophyll, chlorophyllin copper complex or chlorophyllin combined with other laxatives and stool softeners including aloe vera, bisacodyl, cascara, docusate, licorice, buckthorn or senna.

In each of the aforementioned embodiments, the dose of chlorophyll, chlorophyllin copper complex or chlorophylline may range between 1 mg and 300 mg.

In each of the aforementioned embodiments, the total dose of contained ingredients may be between 1 mg to 1500 mg.

### DETAILED DESCRIPTION OF THE INVENTION

The present embodiment presents methods for delivering chlorophyll, chlorophyllin copper complex or chlorophyllin to the rectum and sigmoid colon via a suppository. In so doing, concentrations of these ingredients will be sufficiently high to effect the desired therapeutic actions, while limiting absorption, metabolism and elimination of chlorophyll and chlorophyllin in the small intestine. This method will allow high concentrations of these ingredients in the rectum and sigmoid colon, thus promoting evacuation. An additional benefit of the invention is that it will potentially employ all natural and/or ingredients used for oral preparation in the product. The method also allows for the incorporation of additional substances, such as aloe vera, bisacodyl, cascara, docusate, licorice, buckthorn or senna that may enhance the effectiveness of chlorophyll or chlorophyllin in producing a laxative effect. In addition, because chlorophyll, chlorophylline and chlorophylline copper complex have a green color and may cause discoloration of the skin or underwear in the user, the suppositories contain a thin outer protective layer (also called coating or shell) to prevent such leakage. Finally, the invention utilizes a system to deliver these ingredients directly to the sites of action in the large intestine, thereby avoiding release of these ingredients in the upper gastrointestinal tract. This enables the contents to reach their target at high concentration. Chlorophyll, chlorophyllin copper complex and chlorophyllin also have potential for the treatment of intestinal gas and have fecal deodorant properties.

Theoretically, other laxatives could be used for the intended purposes in place of these ingredients. However, rectally introduced chlorophyll and/or chlorophyllin, with the possible addition of other natural ingredients, has advantage over many other treatments for constipation, namely, greatly reduced or even absence of significant systemic adverse reactions. Rectal chlorophyll, chlorophyllin copper complex and chlorophyllin should have little systemic absorption. Toxicity of chlorophyll, chlorophyllin copper complex and chlorophyllin appears to be limited to photosensitivity. Chlorophyll, chlorophyllin copper complex and chlorophyllin are available as over-the-counter dietary supplements. To date, no suppository containing chlorophyll, chlorophyllin copper complex and/or chlorophyllin have been developed for the treatment of constipation.

The inventors have been able to show that suppositories of chlorophyll copper complex can be produced in a method combined with polyethylene glycol that will not denature the chlorophyll copper complex. This forms a uniform substance with an oily consistency with complete melding of the two individual ingredients. In addition, a method has been developed to produce a thin layer (or shell) can be produced using heated cocoa butter to prevent staining of the skin with chlorophyll copper complex. To date, chlorophyll, chlorophyllin copper complex and chlorophyllin (alone or combined with other substances with laxative properties) have not previously been administered in a suppository form.

### Clinical Experience

These suppositories were tested in three otherwise healthy individuals that had occasional constipation. When placed into the rectum, these subjects found that the suppository completely dissolved, without causing discomfort. After a delay of 15-60 minutes, subjects described a soft, smooth evacuation induced by the suppository. Of note, in one subject the evacuated stool was a green color. This suggests that the chlorophyll copper complexes mixes with fecal material stored in the left side of the colon during the time that the suppository resides in the rectum and sigmoid colon. In the other subjects, green liquid was passed along with normal, firm stool, suggesting that the melted suppository stimulated bowel action, secretion of liquid and defecation. Following melting of the suppository and mixing with colonic contents (either liquid or fecal material or both), the laxative effect of the chlorophyll takes place.

By “suppository,” the invention includes the production of a solid substance that is administered into the rectum that contains medication for anorectal delivery.

Suppositories may be constructed from fatty (or oleaginous) bases and/or water soluble (or miscible) bases.

Fatty bases include the obroma oil (also known as cocoa butter) with or without spermacetic or beeswax to raise the suppository melting point. Additionally, fatty bases for the chlorophyll, chlorophyllin copper complex and/or chlorophyllin suppositories may include synthetic triglycerides and hydrogenated vegetable oils. These may include palm, palm kernel or coconut oils. Name brands utilized for production of vitamin A suppositories may include Fattibase, Wecobee FS, M, R or S, Dehydag, Hydrokote, Suppocire and Witepsol.

Water soluble bases may include glycerated gelatin, with or without preservatives, alginates and polyethylene glycol polymers.

Suppositories may be constructed by hand rolling, compression molding or fusion molding methods.

The basic premise of the present invention is to deliver, in a controlled manner, chlorophyll, chlorophyllin copper complex or chlorophylline directly into the rectum and sigmoid colon to provide a laxative effect. These natural substances hold promise for the treatment constipation due to their laxative properties. Other natural substances may be added to enhance the effectiveness of the combination, including but not limited to aloe vera, bisacodyl, cascara, docusate, licorice, buckthorn and senna. Once the chlorophyll, chlorophyllin copper complex or chlorophylline arrive at the site of action, the ingredients will have a topical effect. This invention also has the potential of alleviating symptoms of secondary causes of constipation such as constipation caused by medications. In addition, it is anticipated that chlorophyll, chlorophyllin copper complex or chlorophylline suppositories may also alleviate symptoms of constipation-predominant IBS including abdominal pain, cramping, bloating and incomplete evacuation.

Chlorophyll, chlorophyllin copper complex and chlorophyllin have been available for some time, as food supplements and natural remedies. They have a long and established safety record. However, the use of these ingredients in a suppository for the treatment of constipation has not been disclosed or suggested by the literature. It is anticipated that because the laxative effects of orally administered chlorophyll, chlorophyllin and chlorophyllin copper complex are mild, large doses of orally administered forms of these substances would be required to achieve desired effects, particularly in patients with moderate or severe constipation. The production of a suppository form of these substances represents a true advance in treatment of constipation as the ingredients are natural and safe and would concentrate in high doses in the rectum to produce a more intense effect than orally administered treatment.

According to a first embodiment, amount of chlorophyll, chlorophyllin copper complex and/or chlorophylline in each suppository is between 1 and 500 mg. The suppository doses will be used up to four times daily. The optimal capsule and tablet sizes and dosages are determined by the results of preliminary studies and can vary with the age, size, and weight of the subject (patient).

According to a first embodiment, the chlorophyll suppository is produced using polyethylene glycol. The suppository has an outer layer that prevents leakage of the chlorophyll or chlorophyllin from the suppository.

The outer layer of the suppository melts upon placement of the suppository into the rectum, releasing the chlorophyll, chlorophyllin copper complex or chlorophyllin contained within the suppository.

The outer layer (or shell) of the suppository may consist of a variety of materials that will retain the chlorophyll, chlorophyllin copper complex or chlorophyllin within the suppository and will dissolve one the suppository is placed in the rectum, thus releasing the active ingredient (s).

The outer covering of the suppository may consist of a variety of fatty (or oleaginous) bases and/or water soluble (or miscible) bases, including cocoa butter, beeswax, synthetic triglycerides, and hydrogenated vegetable oils. (palm, palm kernel or coconut oils). Name brand materials utilized for production of the suppositories or outer layer of the suppositories may include Fattibase, Wecobee FS, M, R or S, Dehydag, Hydrokote, Suppocire and Witepsol.

Water soluble bases to produce the outer portion of the suppository may include glycerated gelatin, or alginates.

Additional pharmaceutically acceptable carriers may be used as stabilizers and can include any and all solvents, dispersion media, coatings, antibacterial and antifungal agents, isotonic, and absorption-delaying agents and the like. The use of such media and agents for pharmaceutically-active substances is well known in the art. Except insofar as any conventional media or agent is compatible with the active ingredient, its use in the therapeutic compositions is contemplated. Supplementary active ingredients also can be incorporated into the compositions.

The compositions and/or methods disclosed and claimed herein can be made and executed without undue experimentation, in light of the present disclosure.

While the compositions and methods of this invention have been described in terms of preferred embodiments, it will be apparent to those of skill in the art that variations may be applied to the compositions and/or methods and in the steps or in the sequence of the steps of the method described herein, without departing from the concept, spirit, and scope of the invention. More specifically, it will be apparent that certain agents which are both chemically and physiologically related may be substituted for the agents described herein, while the same or similar results would be achieved. All such similar substitutes and modifications apparent to those skilled in the art are deemed to be within the spirit, scope, and concept of the invention, as defined by the appended claims.

The references cited herein throughout to the extent that they provide exemplary procedural or other details supplementary to those set forth herein, are all incorporated herein by reference.

What is claimed is:

1. A method of treating constipation, comprising:
a. Providing a suppository containing a pharmacologic dose of chlorophyll, chlorophyllin copper complex or chlorophyllin and an outer covering layer to prevent spillage of active ingredients; and
b. Rectally administering the suppository, wherein the chlorophyll, chlorophyllin copper complex or chlorophyllin will have a direct, topical effect.

2. The method of claim 1, wherein the condition treated is chronic constipation

3. The method of claim 1, where the condition treated is chronic functional constipation.

4. The method of claim 1, where the condition treated is constipation predominant irritable bowel syndrome

5. The method of claim 1, where the disorder is constipation due to a pelvic outlet disorder.

6. The method of claim 1, where the disorder is constipation induced by a variety of diseases.

7. The method of claim 1, where the disorder is constipation due to medications.

8. The method of claim 1, where the disorder is colonic inertia

9. The method of claim 1 in which the suppository or outer layer (or shell) of the suppository is produced by combining chlorophyll, chlorophyllin copper complex or chlorophylline with polyethylene glycol.

10. The method of claim 6 in which the suppository or outer layer (or shell) of the suppository is made from cocoa butter.

11. The method of claim 6 in which the suppository or outer layer (or shell) of the suppository is made from beeswax.

12. The method of claim 6 in which the suppository or outer layer (or shell) of the suppository is made from synthetic triglycerides.

13. The method of claim 6 in which the suppository or outer layer (or shell) of the suppository is made from hydrogenated vegetable oils.

14. The method of claim 1 in which the suppository or outer layer (or shell) of the suppository is made from Wecobee FS, M, R or S, Dehydag, Hydrokote, Suppocire or Witepsol.

15. The method of claim 1 in which the dose of chlorophyll, chlorophyllin copper complex or chlorophylline is between 1 and 500 mg.

16. The method of claim 1 in which the total dose of contained ingredients is 1 mg to 1000 mg.
